(19) United States
(12) Patent Application Publication
(10) Pub. No.: US 2015/0305919 A1
(43) Pub. Date: Oct. 29, 2015

(54) MANDIBULAR ADVANCEMENT DEVICE

(71) Applicant: Airospace Enterprises Pty Ltd, Melbourne (AU)

(72) Inventors: Michael STUBBS, Melbourne (AU); Jack Allen GERSCHMAN, Melbourne (AU); Martin SZWARC, Melbourne (AU)

(21) Appl. No.: 14/702,400

(22) Filed: May 1, 2015

Related U.S. Application Data
(63) Continuation of application No. 12/600,613, filed on Apr. 26, 2010, now abandoned.
(60) Provisional application No. 60/930,841, filed on May 18, 2007.

(30) Foreign Application Priority Data
May 17, 2007 (AU) 2007902628

(51) Int. Cl. A61F 5/56

(52) U.S. Cl. CPC A61F 5/566

(57) ABSTRACT

A mandibular advancement device is proposed which comprises both an upper and a lower base plate, whereby engagement of the plates during closing of the jaws causes protrusion of the mandible and allows relative lateral movement between the jaws. In one particular form the invention relates to a device for positioning upper and lower jaws, including a cam associated with one of the jaws and a follower associated with the other jaw, the follower having an engagement surface which translates across the cam to advance one of the jaws relative to the other as the jaws are closed.

### CROSS-REFERENCE TO RELATED APPLICATIONS

This application is a continuation of U.S. patent application Ser. No. 12/600,613, filed May 19, 2008, which claims priority from Australian Provisional Patent Application No. 2007902628, filed May 17, 2007, and U.S. Patent Application No. 60/930,841, filed May 18, 2007, and which is a U.S. national stage filing of PCT application PCT/AU2008/000697, filed May 19, 2008, the contents of which are incorporated herein by reference.

### BACKGROUND

1. Field of the Invention

This invention relates to a mandibular advancement device, and more particularly, but not exclusively, to a mandibular advancement device which has application in the treatment of Sleep Disordered Breathing, such as snoring, obstructive sleep apnea, upper airway resistance syndrome, and certain Temporomandibular Disorders.

2. Background of the Invention

Mandibular advancement devices endeavor to treat snoring, obstructive sleep apnoea, and upper airway resistance syndrome. A modification of this technology may also be used to treat Temporomandibular Disorders.

There are many different types of mandibular advancement splints currently available. In principal, these devices tend to involve application of a splint to either one arch only (George, 2001) or both dental arches (Pancer et al, 2001; Henke et al, 2000). A typical example has a maxillary arch fitting device with a small acrylic lug that is fitted onto the inferior surface of the maxillary bite plate. The lug engages the lower mandibular incisors which hold the mandible marginally forward, and assists in resolving sleep disordered breathing. Other designs have both the maxillary and mandibular arches engaged with either a spring or screw lock designed metal attachment apparatus which holds the mandible forward. Due to the intensity of the contact force between opposing bite apparatus, most of these mandibular advancement devices often have a risk of occlusal changes. The tendency of many of these devices to fracture over prolonged time, tends to be another unfortunate problem with many mandibular advancement splints. Furthermore, all devices which are currently available have limitations in achieving optimum resolution of sleep disordered breathing.

Examples of the present invention seek to provide a superior mandibular advancement device, and a variation thereof for the treatment of temporomandibular disorders.

### SUMMARY OF THE INVENTION

In accordance with the present invention, there is provided a device for positioning upper and lower jaws, including a cam associated with one of the jaws and a follower associated with the other jaw, the follower having an engagement surface which translates across the cam to advance one of the jaws relative to the other as the jaws are closed.

In accordance with another aspect of the present invention, there is provided a mandibular advancement device including an upper base plate adapted for fitment to an upper jaw of a wearer, and a lower base plate adapted for fitment to a lower jaw of the wearer. The upper base plate has a first cooperating member (in the form of specifically shaped wedges), and the lower base plate has a second cooperating member (also in the form of specifically shaped wedges), wherein the first and second cooperating members interact in response to relative movement between the base plates during closing of the jaws so as to progressively force the lower jaw forward relative to the upper jaw, and wherein the first and second cooperating members are arranged to allow relative lateral movement between the jaws.

In accordance with another aspect of the present invention, there is provided a mandibular advancement device including an upper base plate adapted for fitment to an upper jaw of a wearer, and a lower base plate adapted for fitment to a lower jaw of the wearer. The upper base plate has a first cooperating member, and the lower base plate has a second cooperating member, wherein the first and second cooperating members interact in response to relative movement between the base plates during closing of the jaws so as to progressively force the lower jaw forward relative to the upper jaw, and wherein one cooperating member is in the form of a tubular section extending laterally between the jaws, and the other cooperating member is in the form of a specifically shaped wedges (which could vary between convex, straight or concaved shape) arranged to interact with an outer surface of the tubular section.

In accordance with yet another aspect of the further invention there is provided a mandibular advancement device including an upper base plate adapted for fitment to an upper jaw of a wearer, and a lower base plate adapted for fitment to a lower jaw of the wearer. The upper base plate has incorporates two side plates, which are located on either side of the upper base plate, which has been adapted for fitment to an upper jaw of a wearer. Each of these side plates are shaped so as to push on to an opposing adjustment S mechanism, in the form of two off-centre pins, which are located in the opposing outer side of the lower base plate, which has been adapted to fit the lower jaw of a wearer, and wherein each of the base plates has magnets mounted therein such that a magnetic repulsion between these magnets prevents the dropping of the jaws and enhances the lower jaw protrusion.

Examples of the present invention seek to provide a mandibular advancement device which is adjustable to individual requirements, comfortable to wear, reduces the risk of occlusal changes, and has a low likelihood of fracture, and hence a greater likelihood of longevity, than previously proposed mandibular advancement devices.

Variations of the mandibular advancement device are presented. In each example, the device basically comprises of two components: an upper base plate and a lower base plate, which are fitted to the respective upper and lower jaws. The nature, composition and positioning of the first cooperating member of the plate, in the form of an attachment apparatus on the upper plate, provides engagement surfaces which complement corresponding engagement surfaces of the second cooperating member, in the form of an attachment apparatus of the lower plate, in such a manner that the mandible is kept in a forward position, and prevented from dropping into an inferior and posterior position. In all three examples presented, the advancement of the mandible occurs when both the upper and lower plates each have an attachment which engages into the opposing base, by way of a variety of different cooperating members.

The base plates comprise of close fitting structures, constructed from materials such as acrylic and polycarbonates, which fit snugly over the teeth, permitting a smooth and continuous forward movement of the mandible, while the mandibular advancement device is engaged.

The relative design of the attachment apparatus ensures that mandibular advancement is maintained over the desired range of jaw openings. The angle of inclination of the engaging edges of the attachment apparatus provides a jaw opening path which is generally arcuate with the protrusive border path.

In each example described, the degree of forward mandibular advancement is regulated by the relationship of the contacting surfaces within the attachment apparatus. In all three examples presented, a continuous opening/closing motion of the mouth whilst wearing the device, means that the mandible is pushed forward while still maintaining minimal contact pressure between opposing base plates.

Although the examples of the device are based on the common principle of designing a device in such a manner that the engagement of two surfaces creates and maintains mandibular advancement, the examples differ in relation to the nature and positioning of engagement surfaces attached to the plates, and to some extent, in relation to the location of these plates in the mouth.

These mandibular advancement device options have particular application in the treatment of obstructive sleep apnea and snoring, by allowing the tongue base to be held in a forward position while the device is being worn. The incorporation of magnets, together with the unique configuration of the side plate and the adjustment mechanism in one example also suggests that the device has valuable application in the treatment of temporomandibular disorders, through the reduction of force generated through nocturnal parafunction and bruxism (tooth-grinding) habits, without relying on mandibular advancement.

### BRIEF DESCRIPTION OF THE DRAWINGS

The invention is described, by way of non-limiting example only, with reference to the accompanying drawings in which:

FIG. 1a is a sagittal view of a typical human skull with the mouth wide open, and a mandibular advancement device according to a first example of the invention (“Option 1”) in place in the mouth;

FIG. 1b is a sagittal view of the typical human skull with the mouth in transition towards closing, and the resulting position of the mandibular advancement device of Option 1;

FIG. 1c is a sagittal view of the typical human skull when the mouth is in final resting or optimal closed position, and the resulting position of the mandibular advancement device of Option 1 following full engagement of the attachment apparatus;

FIG. 1d is a sectional elevation view of the attachment apparatus of the plates of the mandibular advancement device of Option 1;

FIG. 1e represents a perspective view of the attachment apparatus of the device of Option 1, showing closer detail of the shape of the driving bridge (2) in the lateral walls (3) of the upper base plate (6), and showing closer detail of the shape of the guiding bridge (1) of the lower base plate (5) of the device;

FIG. 1f is a sagittal view of the typical human skull when the mouth is in final resting or optimal closed position, and the resulting position of the mandibular advancement device of Option 1 with an additional feature in the form of two plastic tubes;

FIG. 2a is a sagittal view of the typical human skull with the mouth wide open, and a mandibular advancement device according to a second example of the invention (“Option 2”) in place in the mouth;

FIG. 2b is a sagittal view of the typical human skull with the mouth in transition towards closing, and the resulting position of the mandibular advancement device of Option 2;

FIG. 2c is a sagittal view of the typical human skull when the mouth is in final resting or optimal closed position, and the resulting position of the mandibular advancement device of Option 2 following full engagement of the attachment apparatus;

FIG. 2d is a perspective view of the upper and lower base plates of the mandibular advancement device of Option 2;

FIG. 2e is a further perspective view of the upper and lower base plates of the mandibular advancement device of Option 2;

FIG. 2f is a sagittal view of the typical human skull when the mouth is in final resting or optimal closed position, and the resulting position of the mandibular advancement device of Option 2 with an additional feature in the form of two plastic tubes;

FIG. 2g is a perspective view of the upper and lower base plates of the mandibular advancement device of Option 2 with an additional feature in the form of two plastic tubes;

FIG. 3a is a sagittal view of the typical human skull with the mouth open, and a mandibular advancement device according to a third example of the invention (“Option 3”) in place in the mouth;

FIG. 3b is a sagittal view of the typical human skull with the mouth in transition towards closing, and the resulting position of the mandibular advancement device of Option 3;

FIG. 3c is a sagittal view of the typical human skull when the mouth is in final resting or optimal closed position, and the resulting position of the mandibular advancement device of Option 3 following full engagement of the attachment apparatus (cooperating members);

FIG. 3d is a sagittal view of the typical human skull when the mouth is in final resting or optimal closed position, and the resulting position of the mandibular advancement device of Option 3 with an additional feature in the form of two tubes;

FIG. 3e is a three-dimensional view of the upper and lower attachment components of the mandibular advancement device of Option 3 when mouth is in an open position;

FIG. 3f is a three-dimensional view of the upper and lower attachment components of the mandibular advancement device of Option 3 when mouth is in a closed position;

FIG. 3g is a front elevation view of the upper and lower attachment components of the mandibular advancement device of Option 3 when the mouth is in a closed position;

FIG. 3h is the side view of the upper and lower attachment components of the mandibular advancement device Option 3, and the location of the side plate and the adjustment mechanism;

FIG. 3i is the detailed configuration of the unique side plate which is attached to the upper attachment components of the mandibular advancement device Option 3;

FIG. 3j is the three-dimensional view showing location of the adjustment mechanism (pin) which is located in the lower attachment components of the mandibur advancement device Option 3; and

FIG. 3k is the three-dimensional view of the components of the side driving plate of the mandibular advancement device Option 3.

### DETAILED DESCRIPTION

Each of the example devices is drawn at three different stages of mouth closure (these being: mouth wide open; mouth partially open; and, mouth closed or at final resting point), to demonstrate the degree of mandibular advancement and changes within the temporomandibular joints that occur during this process.

### Mandibular Advancement Device Option 1—Incorporating Bridges

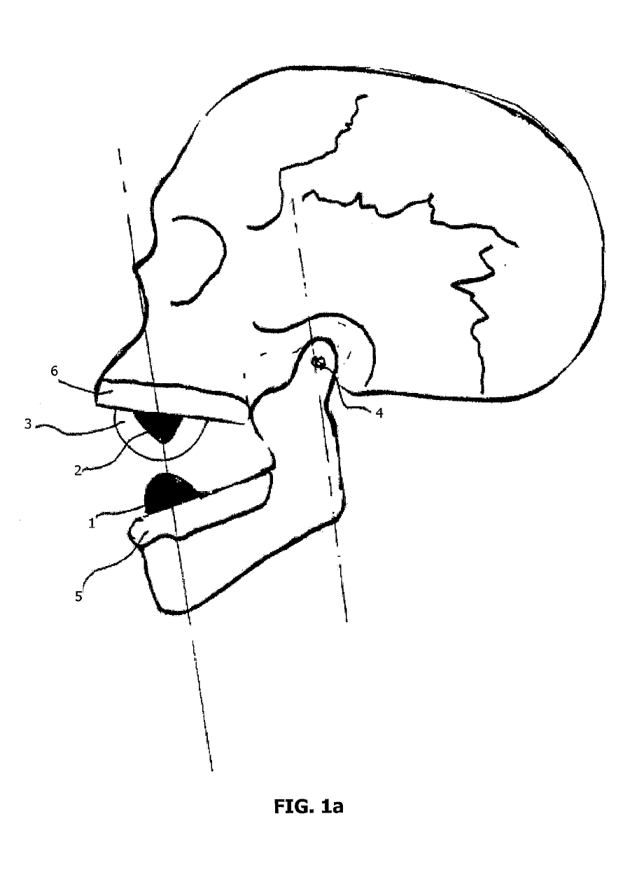

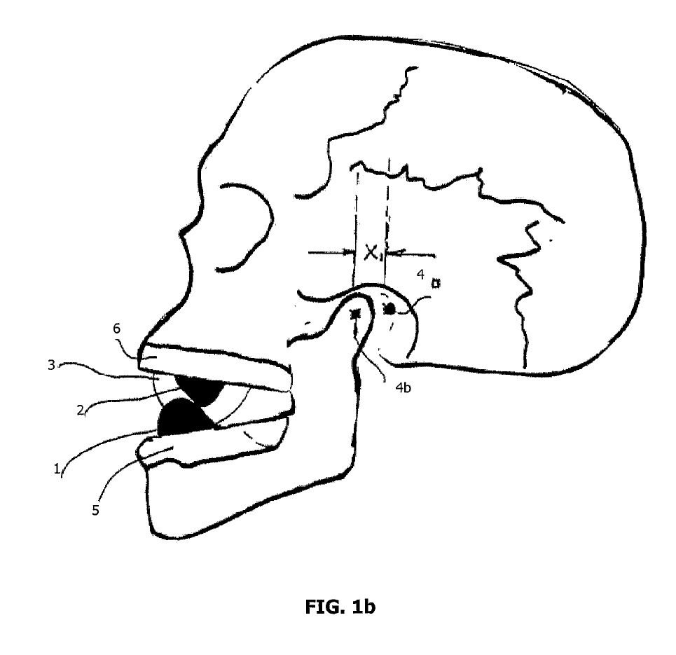

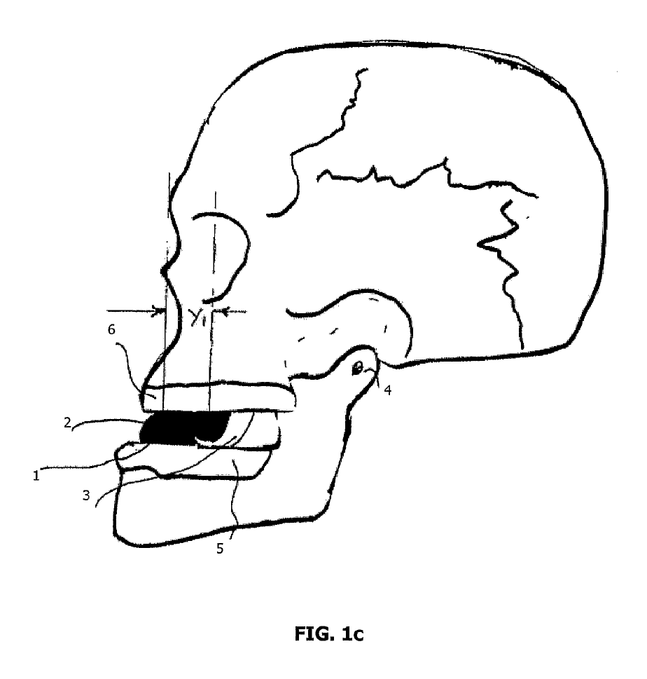

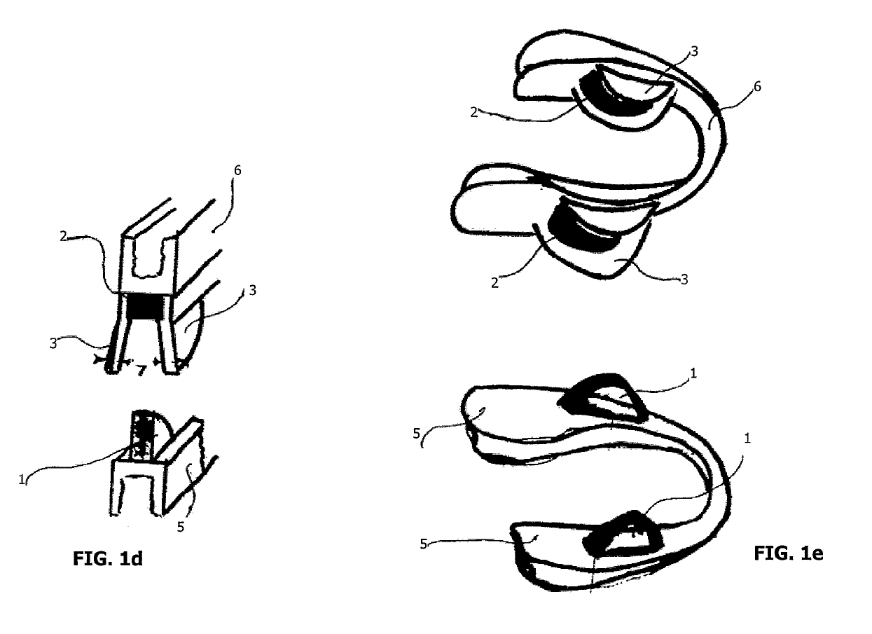

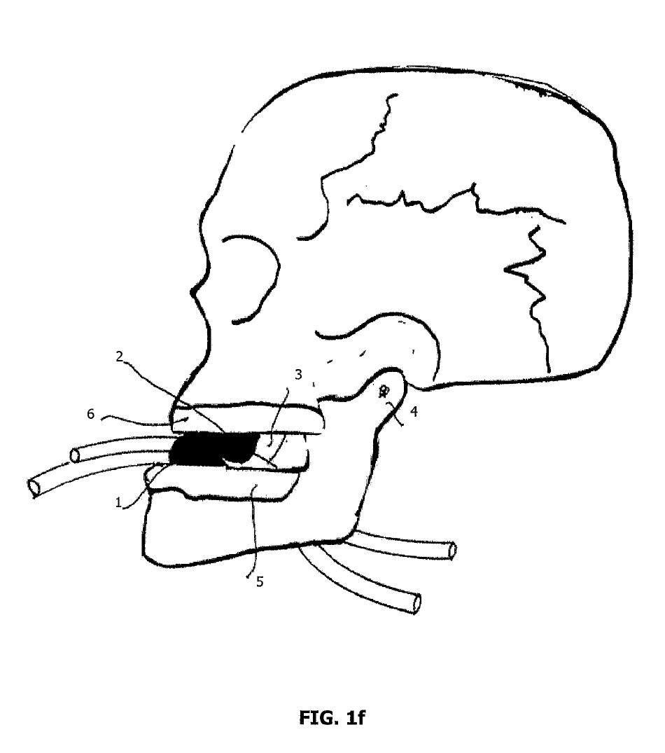

FIG. 1a shows a sagittal view of the typical human skull with the mouth wide open, and the mandibular advancement device Option 1 in place in the mouth.

The various components of this particular device are shown. A cooperating member, in the form of specifically shaped wedges, in this case also referred to as the guiding bridge (1), are attached to the lower base plate (5). Another cooperating member, also in the form of specifically shaped wedges, in this case referred to as the driving bridge (2), sit within lateral barrier walls of the upper attachment apparatus (3), which is attached to the upper base plate (6). In this example, the upper and lower attachment apparatuses are located on the central occlusal surfaces of the posterior teeth.

The attachment apparatus of each of the base plates, in this option, each incorporate two surfaces in the form of specifically shaped wedges (which could vary between convex, straight or concaved shape), and which perform the role of driving and guiding bridges. (The specifically shaped wedges are located on either side of each of the plates, but determination of their exact positioning could vary and may possibly even be positioned in the centre or the outside of these plates).

The condyle in a rotated position (4), hence exhibiting only minimal condylar translation, is also shown.

FIG. 1b shows a sagittal view of the typical human skull with the mouth in transition towards closing, and the resulting position of the mandibular advancement device of Option 1.

As can be seen by this diagram, once the two cooperating member components of the device start engaging and interacting as the mouth starts to close, the mandibular advancement device begins moving the lower jaw part in a forward direction.

The distance change in the condylar position from when the mouth is open (4a), relative to its position after condylar translation (4b), is symbolized in the diagram by X1.

FIG. 1c shows a sagittal view of the typical human skull when the mouth is in final resting or optimal closed position, and the resulting position of the mandibular advancement device of Option 1 following full engagement of the attachment apparatus.

The interaction of the cooperating members, in the form of the driving bridge (2), and the corresponding guiding bridge (1), will cause the mandible to be protruded, as can be clearly seen by the translation or movement of the condyle (4).

The extreme forward displacement of the bottom jaw, in relation to the top jaw, is shown in its final resting or optimal closed position once the mouth is fully closed, and when the attachment apparatus is fully engaged. As this diagram clearly shows, following full engagement of the attachment apparatus, a protruded mandible is the result. This thereby should increase the posterior airway space within the pharynx adjacent to the tongue base, and hence ensure un-hindered airflow into the lungs while the patient is sleeping.

The degree of forward translation of the device can be regulated by the slopes of the specifically shaped wedges (the cooperating members), and thus by the change of the degree of curvature of the guiding and driving members of the device. Hence, the mandible is less forward at position (Y1) on the mandibular attachment arc surface, than when in position (Y2).

FIG. 1d shows a sectional elevation view of the attachment apparatus of the plates of the mandibular advancement device (Option 1).

The lateral walls (3) of the upper base plate are shown to be slightly angled outwardly; the internal distance between the walls is indicated by (7). This flaring assists in easier engaging of wedges forming the driving bridge (2), with the wedges forming the guiding bridge (1). It also allows for a degree of lateral mandibular movement when wearing the device. This flaring will also assist in the more precise positioning of the upper jaw, over the lower jaw, and will also ensure the additional space for prevention of teeth grinding. The sectional elevation view of the driving bridge (2) is also shown. The fitting surfaces of the lower base plate (5), and the fitting surfaces of the upper base plate (6) are also shown. The guiding bridge (1) of the lower base plate engages into the space (7) created between the lateral walls (3) of the upper attachment apparatus.

FIG. 1e represents a perspective view of the attachment apparatus of the device of Option 1, showing closer detail of the shape of the wedges forming the driving bridge (2) in the lateral walls (3) of the upper base plate (6), and showing closer detail of the shape of wedges forming the guiding bridge (1) of the lower base plate (5) of the device.

FIG. 1f shows a sagittal view of the typical human skull when the mouth is in final resting or optimal closed position, and the resulting position of the mandibular advancement device of Option 1 with an additional feature in the form of two tubes, for example, for the purpose of delivery of oxygen and suction of oral fluids,

A circular tube is incorporated within each base plate, this location being within the lingual surface of the lower base plate (5), and the palatal surface of the upper base plate (6), respectively. Each tube has multiple perforations.

The engagement of the attachment devices, in the form of specifically shaped wedges forming the driving bridge (2), and specifically shaped wedges forming the corresponding guiding bridge (1), will cause the mandible to be protruded, as evidenced by the translation or movement of the condyle (4).

The lower base plate has a small circular tube embedded and exiting within the mid-line of the base plate and can be attached to a suction device. The upper base plate has similar small circular tube embedded within the palatal surface of the upper base plate, and exits the midline which can be attached to an oxygen source.

Incorporation of these tubes (for suction and oxygen delivery purposes) can permit application of these devices in maintaining the patency of a patient's airway during postoperative recovery following general anaesthesia, or where paramedical treatment, following patient trauma, has occurred.

Further modification of this unique tubing arrangement is proposed, in which two circular corresponding tubes, comprising of one tube located within a second outer tube, are embedded within each of the base plates. Each outer tube will have numerous small perforations. These perforations will act as components of a suction device. Simultaneously, each of the inner tubes will be utilized in the delivery of oxygen, which will be able to be released at the end of both tubes, at the distal margins of each base plate.

### Mandibular Advancement Device Option 2—Incorporating a Tubular Section

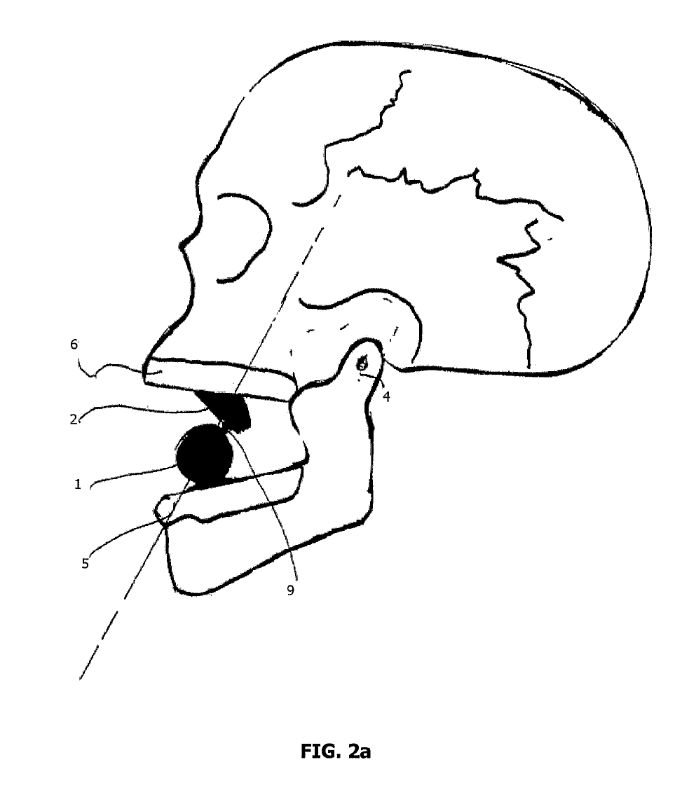

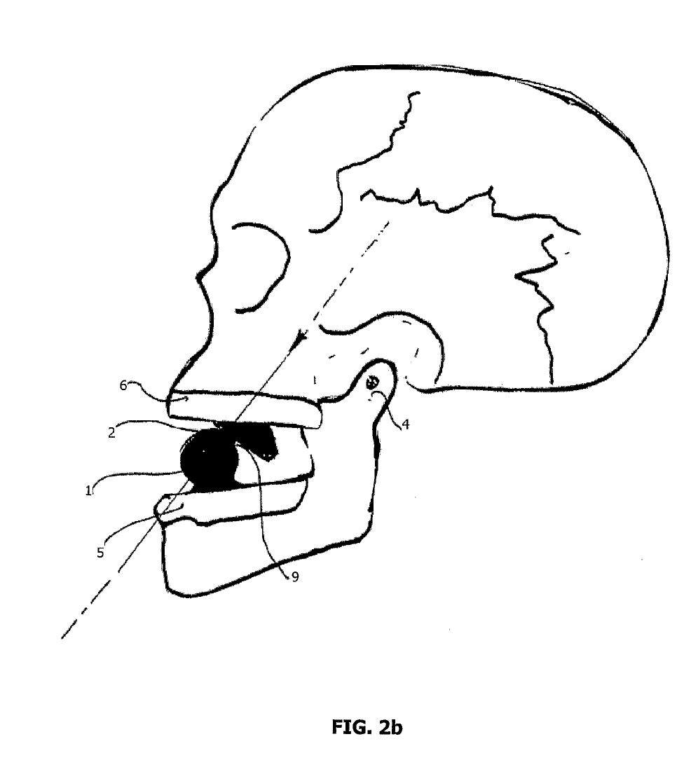

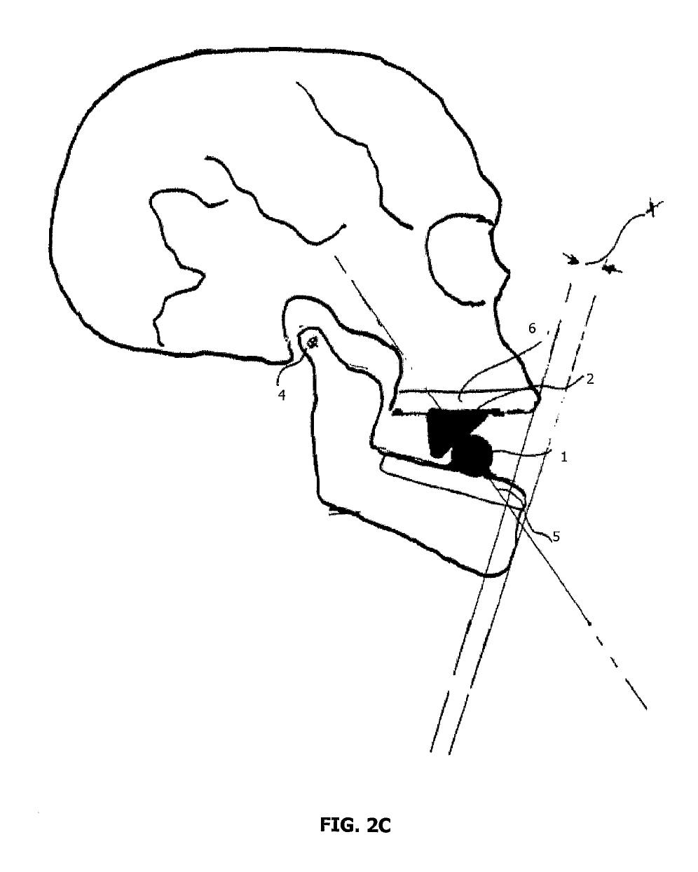

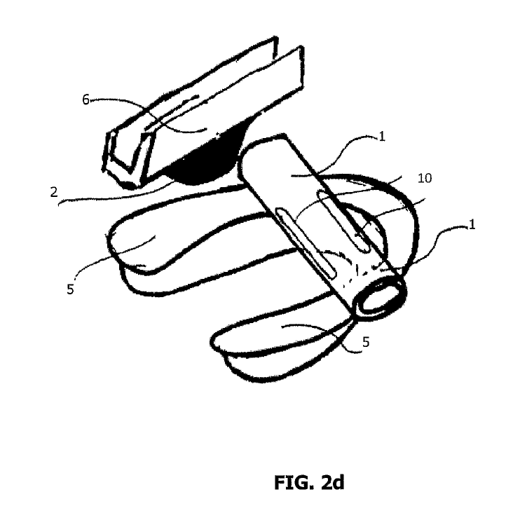

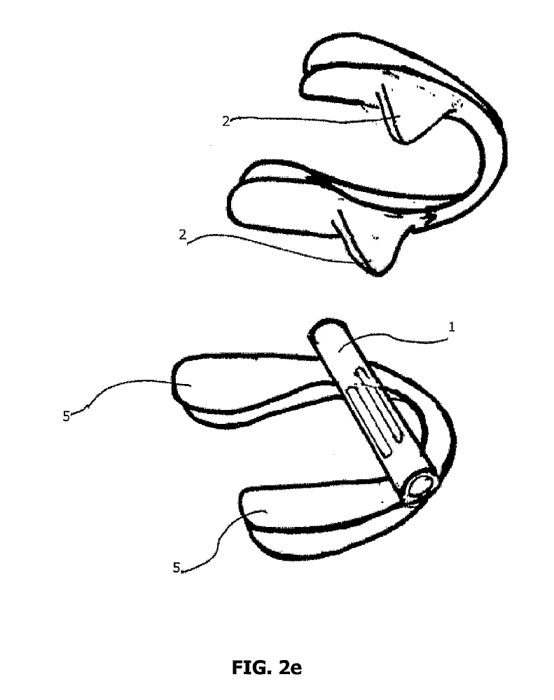

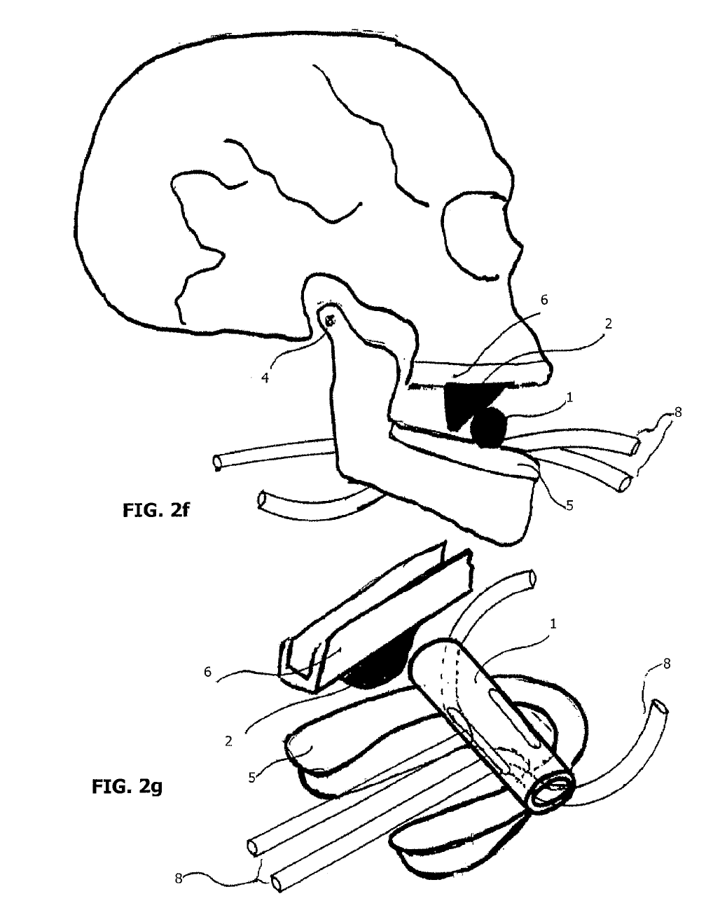

FIG. 2a shows a sagittal view of the typical human skull with the mouth wide open, and the mandibular advancement device of Option 2 in place in the mouth.

The various components of this particular device are shown. The attachment apparatus of the upper base plate (6), in this option, incorporates two surfaces in the form of specifically shaped wedges (2) (which could vary between convex, straight or concaved shape) and which perform the role of the driving bridge. (The specifically shaped wedges are located on either side of the plate, but determination of their exact positioning could vary and may possibly even be positioned in the centre or the outside of this upper plate). The wedges project downwards and, as the mouth closes, will engage with the lower attachment apparatus which has a tubular section (1) which is attached to the lower base plate (5). The tubular section performs the role of a guiding bridge. The tubular section can be moved to different positions on the lower base plate (in order to vary the degree of forward mandibular movement, based on individual requirements). The specifically shaped wedges (2) engage the lower attachment apparatus (1) at an obtuse contact angle (9), which will drive the mandible forward upon contact. In this device option, the attachment apparatus of the device are located anteriorly, along the plane aligning the lower canines.

The condyle in a rotated position (4), hence exhibiting only minimal condylar translation, is also shown.

The straight line shows the angle of contact of the specifically shaped wedges (2) on the tubular section (1) when the mouth is in open position. It also shows the extent of the angle between the contact point on the tube by the upper jaw, relative to the vertical axis through the centre of the tube.

FIG. 2b shows a sagittal view of the typical human skull with the mouth in transition towards closing, and the resulting position of the mandibular advancement device of Option 2,

The attachment apparatus, in the form of specifically shaped wedges (2), is contacting the lower base plate attachment apparatus, in the form of a tubular section (1), As can be seen by this diagram, once the two attachment components of the device start engaging as the mouth starts to close, the mandibular advancement device is now in a forward moving position, Hence, as demonstrated by the slant of the straight line in the diagram, the angle of contact between the two cooperating members has changed from that shown in FIG. 2a. The mandible is now driven further forward with much less force compared to the initial contact. The new condylar position at this point is shown (4).

FIG. 2c shows a sagittal view of the typical human skull when the mouth is in final resting or optimal closed position, and the resulting position of the mandibular advancement device of Option 2 following full engagement of the attachment apparatus.

The engagement of the attachment apparatus, in the form of the specifically shaped wedges (2), and the corresponding surface incorporating a tubular section (1), will cause the mandible to be protruded, as evidenced by the translation or movement of the condyle (4).

The extreme forward displacement of the bottom jaw, in relation to the top jaw, is shown in its final resting or optimal closed position once the mouth is fully closed, and when the attachment apparatus is fully engaged. As this diagram clearly shows, following full engagement of the attachment apparatus, a protruded mandible is the result.

The extent of forward movement (x) of the mandible that occurs at this closed position, when optimal contact between the two opposing attachment devices within the base plates takes place, is shown. The forward translation of the condylar head during optimal engagement of both mandibular components is also illustrated (4).

FIG. 2d shows the perspective view of the upper and lower base plates of the mandibular advancement device Option 2. The diagram shows a cross-section of the attachment apparatus (2), in this version being the wedges on the upper base plate (6), and the lower attachment apparatus, in this version being the tubular section (1). The detailed view of the lower base plate (5) with the tubular section (1), in which a number of linear perforated slots (10) are incorporated, is also shown. The perforated linear slots (10) will enhance the airflow into the oral cavity at all stages of the device movement.

FIG. 2e shows a further perspective view of the upper and lower base plates of the mandibular advancement device of Option 2. The diagram shows further details of the upper attachment apparatus, indicating an example of the positioning of the two wedges (2) on the upper base plate (6), and the lower attachment apparatus, in this version being the tubular section (1).

The positioning of the wedges can be set anywhere under the upper plate. In some cases, it might even be necessary to position these wedges on the extreme sides of the upper plate, depending on individual requirements and comfort.

The amount of forward displacement or mandibular forward travel can be regulated by the curvature or change of shape of the contact surface of the driving wedges. The change of shape will alter the ratio of downward travel of the upper jaw, in relation to the lower jaw, while the lower jaw is being pushed forward. In some cases, even a slight change in the shape of the circumference of the tubular section into a different shape (for example, an oval shape), can further enhance the performance of the device.

The unique design of the contact surfaces of the cooperating members eases the force needed to push the lower jaw forward once the process is half way in downward motion. Pushing down on the cylindrical shape of the tubular section at a slight angle to its axis will require a certain effort for it to move in a side direction; but once the force is moved towards the lower part of the curved surface, the force required is only a fraction of the original applied force. This unique combination of unrestricted jaw movement will also allow total lateral movement, thereby reducing teeth grinding and clenching.

FIG. 2f shows a sagittal view of the typical human skull when the mouth is in final resting or optimal closed position, and the resulting position of the mandibular advancement device of Option 2 with an additional feature in the form of two plastic tubes.

A tube (8) is incorporated within each base plate, this location being within the lingual surface of the lower base plate (5), and the palatal surface of the upper base plate (6), respectively. Each tube comprises of a small circular tube with multiple perforations.

The lower base plate (5) has the suction tube exiting within the mid-line of its base and can be attached to a suction device, The upper base plate (6) has a similar small circular tube embedded within the palatal surface of the upper base plate and exits the midline which attaches to an oxygen source.

As in previous versions, the engagement of the attachment devices, in this case, in the form of the specifically shaped wedges (2), and the corresponding tubular section (1), will cause the mandible to be protruded, as evidenced by the translation or movement of the condyle (4).

The lower base plate has a small circular tube embedded and exiting within the mid-line of the base plate and can be attached to a suction device. The upper base plate has a similar small circular tube embedded within the palatal surface of the upper base plate, and exits the midline which can be attached to an oxygen source.

Incorporation of these tubes (for suction and oxygen delivery purposes) can permit application of these devices in maintaining the patency of a patients' airway during postoperative recovery following general anaesthesia, or where paramedical treatment, following patient trauma, has occurred.

Further modification of this unique tubing arrangement is proposed, in which two circular corresponding tubes, comprising of one tube located within a second outer tube, are embedded within each of the base plates. Each outer tube will have numerous small perforations. These perforations will act as components of a suction device. Simultaneously, each of the inner tubes will be utilized in the delivery of oxygen, which will be able to be released at the end of both tubes, at the distal margins of each base plate,

FIG. 2g shows the perspective view of the upper and lower base plates of the mandibular advancement device of Option 2 with an additional feature in the form of two plastic tubes.

### Mandibular Advancement Option 3—Incorporating Magnets and the Unique Configuration of the Side Plate and the Adjustment Mechanism

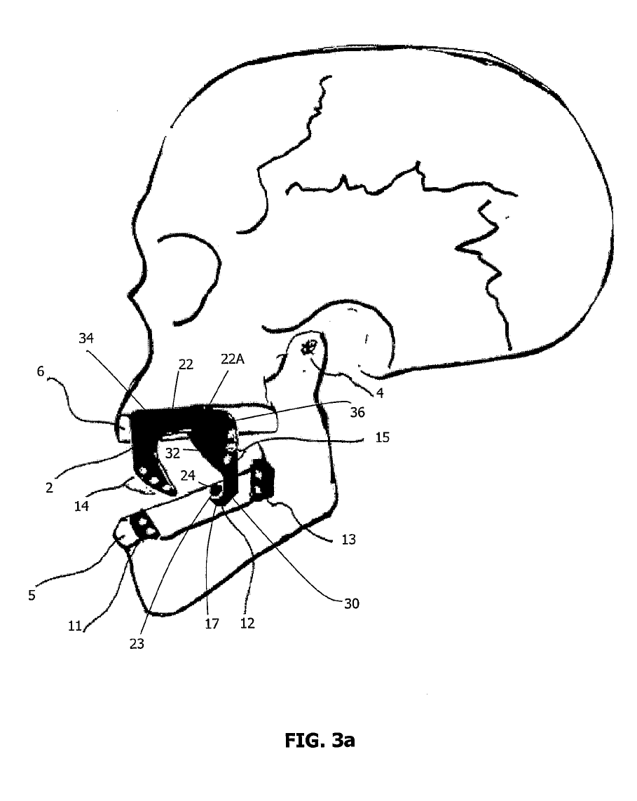

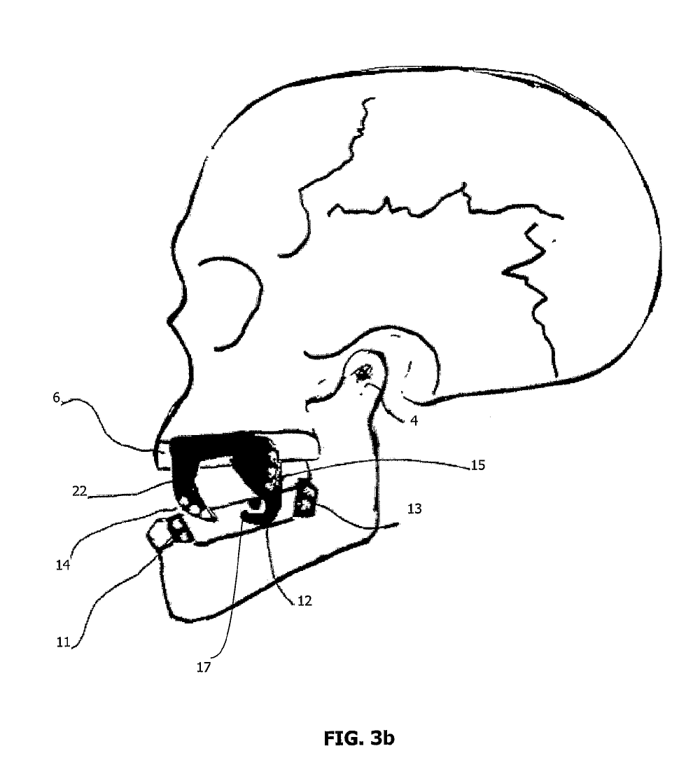

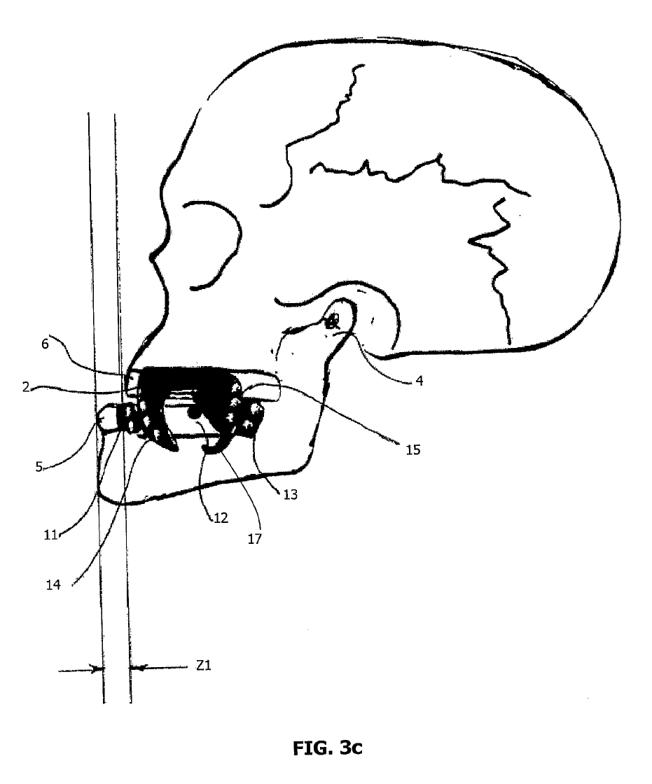

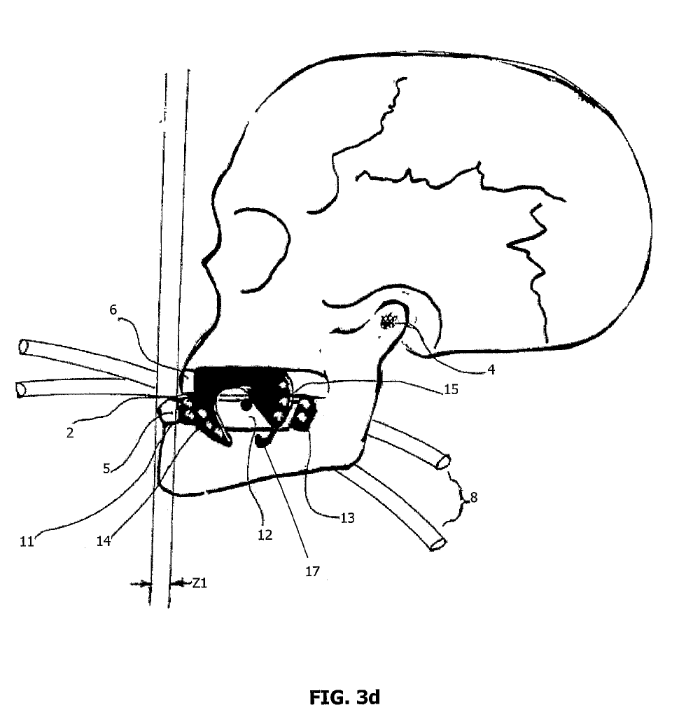

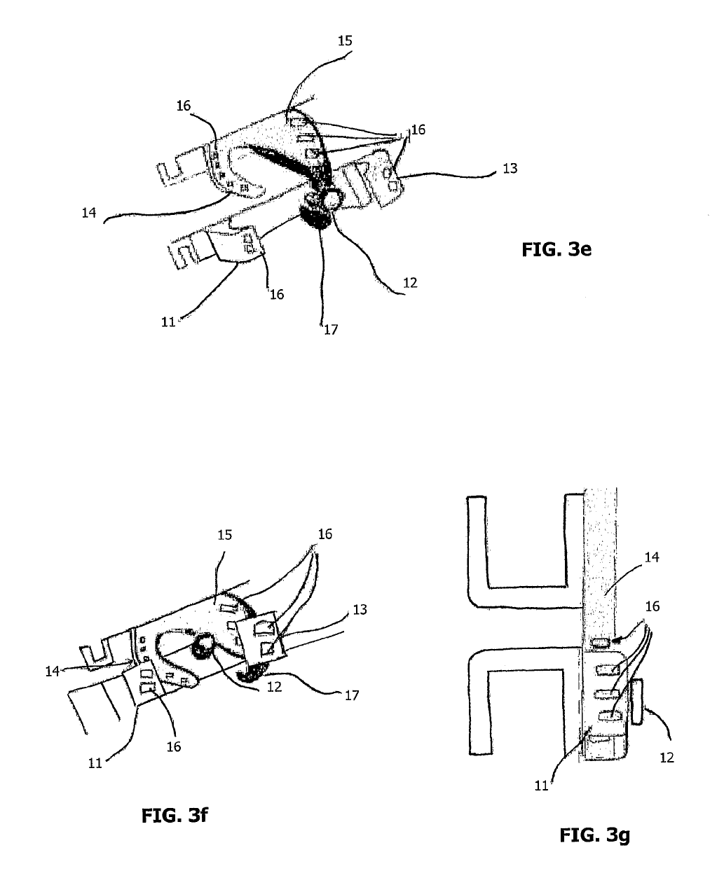

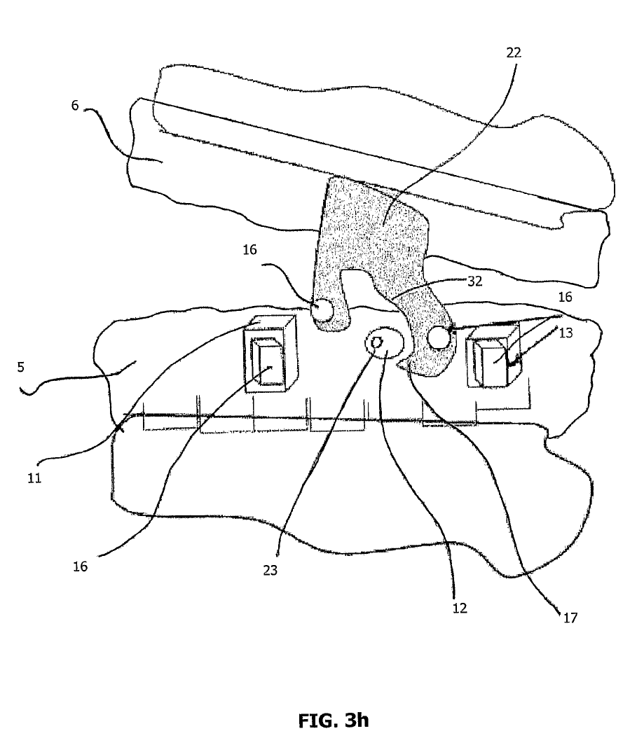

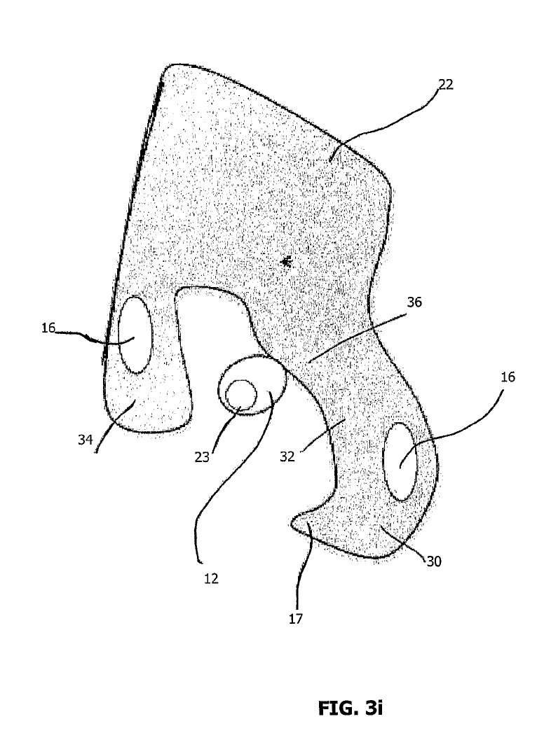

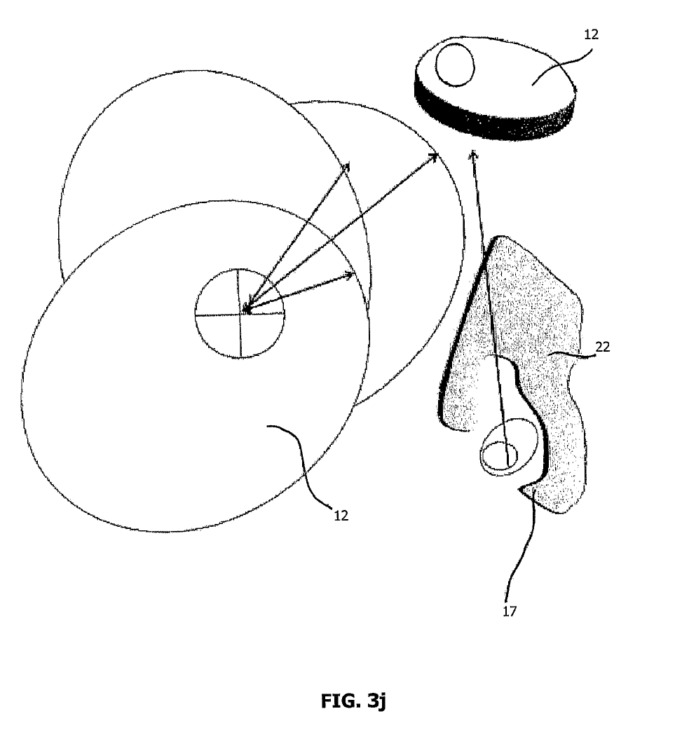

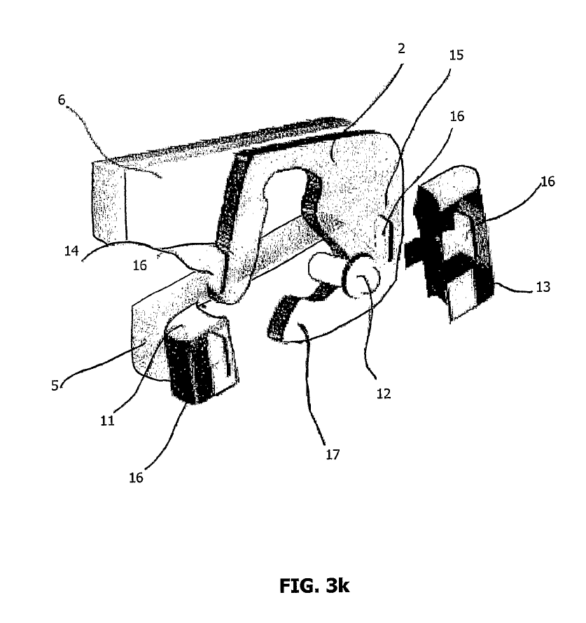

FIG. 3a shows a sagittal view of the typical human skull with the mouth open, and the mandibular advancement device of Option 3 in place in the mouth.

The various components of this particular device are shown. The use of magnets and the unique configuration of the side plate and the adjustment mechanism are the key to this particular option of the mandibular advancement device. A number of magnets are fully embedded in a casing in the base plates (5) and (6) of the device.

In this version, two side plates (22), are located on either side of the upper base plate 6, Each of these side plates 22 function as followers (22A) are shaped so as to push on to an opposing cam 23 in the form of an adjustment mechanism 24, provided by two off-centre pins (12), which are located in the opposing outer side of the lower base plate (5), which has been adapted to fit the lower jaw of a wearer. The purpose of the pins (12) and followers (22A) is to guide the upper plates (6) relative to the lower plates (5) as the jaws are closed.

The inner curvature of each of these side plates (22), has an alternating convex and concave surface. In particular, the follower 22A has a first section 30 which defines a first curved path forming part of the engagement surface 32 which contacts the cam 23 when the jaws are open. A second section 34 defines a second curved path which forms part of the engagement surface 32 when the jaws are closed together. A third section 36 is provided which defines a third curved path that forms part of the engagement surface that transitions between the first and second curved paths. The third curved path is ramped toward the second curved path to effect progressive advancement of the lower jaws as the jaws are closed together. Accordingly, when the curved engagement surface comes in contact with the opposing cam adjustment mechanism (the pin (12)), the lower jaw will be propelled forward. This resulting mandibular advancement of the lower base plate is of critical importance, in that it opens the airway and allows the breathing capacity and air intake to be increased (as also demonstrated in FIG. 3b.

This factor is of great importance in the treatment of Sleep Disordered Breathing, such as snoring, obstructive sleep apnea, and upper airway resistance syndrome. This novel design also has application in the treatment of Temporomandibular Disorders.

This effect of mandibular advancement of the lower jaw is achieved by the fact that the engagement or contact surface of the follower (22A) of the upper base plate (5) creates a forward force on the opposing adjustment mechanism (the pin (12)). In turn, the downward force by the side plate during the closure of the mouth, and its subsequent contact with the opposing pin, will force the lower jaw to have no other alternative but to be pushed forward in such a manner that the mandible is prevented from dropping into an inferior and posterior position.

The unique feature of the design of this side plate is that the amount of curvature—starting from straight line to a severe radius—can be altered to individual needs, because the ratio of the required amount of forward travel, in relation to the downward travel of the upper jaw, can be controlled and adjusted. Basing the design of this device on this unique relationship between the off centre pin and opposing curved surfaces in the design of the driving plate, is of major innovation.

There is also a hook (17) designed to function as a buffer within the lower part of the side plate. This hook will assist in preventing the two base plates (upper and lower) from separating once inserted in the mouth. This is a very important feature of this mandibular advancement splint, since it will stop, or eliminate, the tendency of the lower jaw disengaging out of the splint, which in turn will prevent the splint falling downwards and/or avoid the problem of closing the airways, thereby optimizing patient compliance.

The adjustment mechanism (the off-centre pin) further enhances the performance of this device. The ovoid shape of the pin (12), once rotated, will alter the distance between the axis of the pin, and the contact point of the plate (22). This means that the starting position of the travel of the lower jaw, can be controlled at more precise increments, than in prior mandibular advancement devices, hence the device can be fine-tuned more successfully for best performance, and be specifically tailored for different occlusions and different shaped dental arches. This fine-tuning and the top performance of the mandibular advancement splint is achieved by the anticlockwise rotation and locking (against reverse movement) of the opposing adjustment pin.

Because the pin (12) is oval shaped, rather than round, and is of varying circumference, the first contact points of the two base plates of the splint can be easily altered and/or adjusted, at any stage, hence providing a mandibular advancement device which is adjustable to individual requirements.

This flexibility of fitting enables the attainment of greater forward travel of the lower jaw. The opposing adjustment pin can be rotated in a way that will enable greater forward movement of the lower jaw (mandibular protrusion), if required.

The unique locking of the adjustment pin in an anticlockwise rotation, ensures that any downward force of the upper base plate on the pin (12) prevents it from rotating in an anti-clockwise direction. The fact that the pin cannot rotate in an anti-clockwise direction facilitates optimal control over the required degree of lower jaw protrusion.

The outer surface of the lower base plate (5) is designed to house a number of magnets that will perform a spongy low resistance movement of the splint. The magnetic forces created by the inserted magnets will propel the movement of this device, thereby enhancing its performance. These magnets are embedded completely within the casing and so are not exposed directly into the oral cavity.

The condyle in a rotated, position (4) with minimal condylar translation, is also shown,

The embedded magnets can be located within both the upper and the lower base plates (5) are shown. These magnets generate an opposing force to similar magnets which are embedded in the attachment apparatus of the lower base plate (11). The magnets on the distal surface (15) of the upper attachment apparatus are also embedded completely within the casing, and thereby not exposed directly into the oral cavity. These generate an opposing force to similar magnets (7) which are seen to be embedded in the attachment apparatus of the lower base plate on both the proximal (11) and distal (13) surfaces of the plate.

The positioning of the magnets within the upper and lower part of the splint and their polarities will vary for some individual cases. It is quite possible that the opposing forces of the magnets might be reversed and used as attracting forces in some individual applications. It is also possible to place magnets on the flat inner surfaces of the upper and lower splints.

FIG. 3b shows a sagittal view of the typical human skull with the mouth in transition towards closing, and the resulting position of the mandibular advancement device Option 3 associated with both the unique feature of the design of the side plate, adjustment pin and placement of magnets.

The positioning of magnets, together with the unique configuration of the side plate (including the buffer hook) and the adjustment mechanism is shown.

When the mouth is in transition towards closing, the pin (12) contacts the distal surface of the upper attachment apparatus which starts the protrusive movement of the mandible. As the mouth continues to close, the generation of opposing magnetic forces occurs, The front lower magnets (11) commence to generate an opposing force to the magnets in the front upper magnetic compartment (14), while the rear lower magnets (13) also commence to generate an opposing force to the magnets in the rear upper magnetic compartment (15). The clinical effect of this engagement causes pushing upwards, which starts to partially open the mouth.

FIG. 3c shows a sagittal view of the typical human skull when the mouth is in final resting or optimal closed position, and the resulting position of the mandibular advancement device Option 3 following full engagement of the attachment apparatus.

The positioning of magnets, together with the unique configuration of the side plate (including the buffer hook) and the adjustment mechanism is shown. The extent of forward displacement of the mandible is represented by Z1. The degree of forward mandibular movement is regulated by the contact between the guiding pin (12), and the contact distal surface angle of the upper attachment apparatus (2).

The amount of transnational movement of the condylar head during optimal contact of both attachment apparatus is represented.

The overall effect of this contact between upper and lower attachment apparatus in this particular version of the device is a unique ‘spongy effect’ in which the closer the base plates engage, the more the magnets push the jaw open, due to the same polarity of the magnets. The magnets are situated in a manner such that same polarity (S) will always face the repelling force of the approaching surface. This constant repetitive clenching and relaxation of the jaw pushes the mandible forward and permits a brief period of relaxation of the forward projection of the mandible, before re-engagement of the attachment apparatus, and recommencement of mandibular protrusion.

This low force of contact between opposing base plates will lower the risk of occlusal changes often seen with most mandibular advancement devices. It will reduce the degree of occlusal parafunctional forces on the device base plates.

FIG. 3d shows a sagittal view of the typical human skull when the mouth is in final resting or optimal closed position, and the resulting position of the mandibular advancement device of Option 3 with an additional feature in the form of two plastic tubes.

A small circular tube (8) is incorporated within each base plate. These tubes are located within the lingual surface of the lower base plate, and the palatal surface of the upper base plate, respectively. Each of these small circular tubes has multiple perforations.

The lower base plate has the suction tube exiting within the midline of its base plate, and will be attached to a suction device. The upper base plate has a similar small circular tube embedded within its palatal surface, and exits the midline which attaches to an oxygen source.

The engagement of the unique attachment apparatus, with the aid of the guiding pin (12) and the assistance of the repelling force of the magnetic field, will cause the mandible to be protruded (as evidenced by the translation or movement of the condyle (4)).

Further modification of this unique tubing arrangement is proposed, in which two circular corresponding tubes, comprising of one tube located within a second outer tube, are embedded within each of the base plates. Each outer tube will have numerous small perforations. These perforations will facilitate the suction action of the device.

Simultaneously, each of the inner tubes will be utilized in the delivery of oxygen, which will be able to be released at the end of both tubes, at the distal margins of each base plate.

FIG. 3e shows a three-dimensional view of the upper and lower attachment components of mandibular advancement device Option 3 when mouth is in an open position.

FIG. 3f shows a three-dimensional view of the upper and lower attachment components of mandibular advancement device Option 3 when mouth is in a closed position.

FIG. 3g shows a front elevation view of the upper and lower attachment components of mandibular advancement device Option 3 when mouth is in a closed position.

FIG. 3h shows the side view of the upper and lower attachment components of the mandibular advancement device Option 3, and the location of the side driving plate (22) and the adjustment mechanism/pin (12).

The positioning of magnets (13) and their casing (16), together with the unique configuration of the side plate, including the buffer hook 17, and the adjustment mechanism (12) is shown.

As described above, the inner curvature of each of these side plates (22), has an engagement surface 32 that includes an alternating convex and concave surface. When this curvature comes in contact with the opposing cam 23/adjustment mechanism (the pin (12)), the lower jaw will be propelled forward. This resulting mandibular advancement of the lower base plate is of critical importance, in that it opens the airway and allows the breathing capacity and air intake to be increased.

FIG. 3i shows the detailed configuration of the unique follower (22A)/side driving plate (22) which is located on the upper attachment components of the mandibular advancement device Option 3. The location of the cam (23)/adjustment pin (12), in contact with the engagement surface (32) of the inner part of the upper attachment apparatus is shown. The proposed location of the magnets (16) is also shown.

The unique feature of the design of this side plate is that the amount of curvature—starting from straight line to a severe radius—can be altered to individual needs, because the ratio of the required amount of forward travel, in relation to the downward travel of the upper jaw, can be controlled and adjusted. Basing the design of this device on this unique relationship between the off centre pin and opposing curved surfaces in the design of the driving plate, is of major innovation.

FIG. 3j shows the three-dimensional view showing location of the adjustment mechanism (pin) which is located in the lower attachment components of the mandibular advancement device Option 3. The location of the driving plate (22) in relation to the guiding pin (12) when in contact is also shown. The varying distances from the axis point to the point of contact with the driving plate due to the rotation and adjustment of the pin, are illustrated.

The oval shape of the pin (12), once rotated, will alter the distance between the axis of the pin, and the contact point of the upper base plate. This means that the starting position of the travel of the lower jaw, can be controlled at more precise increments, than in prior mandibular advancement devices, hence the device can be fine-tuned more successfully for best performance, and be specifically tailored for different occlusions and different shaped dental arches.

FIG. 3k is the three-dimensional view of the components of the side driving plate of the mandibular advancement device Option 3. The possible location of the magnet compartments (11), and (13) on the lower base plate, and the magnetic compartments within side driving plate (22) of the upper base plate are also shown. In this instance, the device is in fully open position, showing the adjustment mechanism in the form of the unique off-centre guiding pin (12) engaged in the lowest point of the upper base driving plate (2). This design ensures that the driving plate (2) and the buffer hook (17) restricts the lower jaw from falling backwards.

Advantageously, examples of the present invention provide a range of mandibular advancement devices, each of which share the common benefits of being adjustable to individual requirements, while at the same time, having maximum comfort, greatly reduced risk of occlusal changes (by having a unique low contact force between opposing bite plates), low likelihood of fracture, and hence a greater likelihood of longevity than previously designed mandibular advancement devices.

Incorporated within the design of all options of these mandibular advancement devices is the possibility of some lateral mandibular excursions. This is important with regard to both maintaining patient comfort, and reduced shear stresses on the attachment apparatus itself.

The newly proposed mandibular advancement device options arc designed with the intention of protruding the mandible up to the optimal protrusive position possible. This is created in order to increase the posterior airway space within the pharynx adjacent to the tongue base, and hence ensure unhindered airflow into the lungs while the patient is sleeping.

The cooperating member attachment apparatus of each of the device options are located either on each base plate within the central occlusal surfaces of the posterior teeth (Option 1) or anteriorly, along the plane aligning the lower canines (Option 2), or on the buccal surfaces of the posterior teeth (Option 3).

Each device option also has further optional components, in the form of a tube for the delivery of oxygen, which is incorporated into the upper base plate, and a suction tube that is incorporated into the lower base plate. This additional component permits application of these devices in maintaining the patency of a patient's airway during postoperative recovery following general anesthesia, or where paramedical treatment following patient trauma has occurred.

The attachment apparatus mechanisms of each device option are adjustable, at least during manufacture, and adjustments are dependent on the biting surface of the device that drives the mandible forward. Hence, the degree of protrusive movement is guided by the angle and shape of the contact surfaces between opposing attachment apparatus within each base plate.

In one particular device which utilizes magnets together with the unique configuration of the side plate (including the buffer hook) and the adjustment mechanism (Option 3), mandibular advancement is achieved when both the upper and lower base plate components are engaged during mouth closure. Each of these side plates is shaped so as to push on to an opposing cam adjustment mechanism. Each of the upper and lower base plate components has magnets mounted within, to create a magnetic repulsion between these magnets which prevents the dropping of the jaws and enhances the lower jaw protrusion. Such engagement initiates a magnetic repulsion force from the various magnets placed within the attachment apparatus. This creates a unique low contact force between opposing bite plates, and the constant working of the jaw by closure, and then reflex partial opening due to the magnetic repulsion. It is postulated that this unique mechanism will lower the risk of occlusal changes often seen with most mandibular advancement devices. This low force of contact between opposing base plates will reduce the degree of occlusal parafunctional forces on the plates. This unique configuration of the side plate (including the buffer hook) and the adjustment mechanism and the Inclusion of magnets will also enhance the longevity of the device reducing the likelihood of fracture, thereby preventing a common problem with many mandibular advancement splints. These particular features also have applications in the treatment of temporomandibular disorders, by reducing the effective generation of pressure from nocturnal occlusal parafunctional habits.

Incorporated within the design of all three mandibular advancement device options is the possibility of some lateral mandibular excursions. This is important with regard to both patient comfort, and reduced shear stresses on the attachment apparatus itself.

Each mandibular advancement device option also will be structured in a manner such as to ensure unrestricted vertical dimensional opening between opposing base plates. This will enable the degree of airflow to be controlled, thereby Optimizing patient compliance.

Furthermore, in circumstances where occlusal vertical dimension is excessive, this feature will also reduce the likelihood of distalisation of the tongue base.

While an example of the present invention has been described above, it should be understood that it has been presented by way of example only, and not by way of limitation. It will be apparent to a person skilled in the relevant area that various changes in form and detail can be made therein without departing from the spirit and scope of the invention. Thus, the present invention should not be limited by the above described example.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or Step or group of integers or steps.

### DIAGRAM LEGEND

1 Attachment apparatus on the lower base plate (guiding bridge)
2 Attachment apparatus on the upper base plate (driving bridge/plate)
3 Lateral walls of the attachment apparatus on the upper base plate
4 Condyle in a rotated position
4a Condylar position when the mouth is open
4b Condylar position after condylar translation
5 Lower base plate
6 Upper base plate
7 Distance between the lateral walls
8 Plastic tube
9 Contact point between the wedge and tube
10 Perforated slots in the tube
11 Front lower magnetic compartment
12 Guiding pin in the lower base plate
13 Rear lower magnetic compartment
14 Front upper magnetic compartment
15 Rear upper magnetic compartment
16 Magnets
17 Buffer Hook
22 Plate
22A Follower
23 Cam
24 Adjustment Mechanism
30 First section
32 Engagement surface
34 Second section
36 Third section
X1 represents the change in distance of the forward movement of the condyle upon the month closing and engaging of the attachment apparatus (i.e. the change in distance) between 4b and 4.
Y1 represents the change in the full forward distance of the bottom jaw, in relation to its starting position (when the mouth was fully open).
Z1 represents the extent of forward displacement of the mandible.

I/we claim:

1. A device for positioning upper and lower jaws, including a pin associated with one of the jaws and a follower associated with the other jaw, the follower having an engagement surface which moves over the pin to advance a first of the jaws relative to a second of the jaws when the jaws are closed;
wherein the pin is adapted such that rotation of the pin allows for adjustment of the distance by which the first jaw is advanced relative to the second jaw, and
wherein the device is configured such that the operation of the device is driven by the movement of the jaws as the jaws are closed.

2. The device of claim 1, wherein the pin has an ovoid cross-section.

3. The device of claim 1, wherein the pin is arranged to be rotated in one direction only, for adjustment.

4. The device of claim 1, wherein the follower includes:
a first section which defines a first curved path forming part of the engagement surface associated with movement when the jaws are open;
a second section which defines a second curved path forming part of the engagement surface associated with the jaws when closed together; and
a third section defining a third curved path that forms part of the engagement surface that transitions between the first and second curved paths, the third curved path being ramped toward the second curved path to effect progressive advancement of the first jaw as the jaws are closed together.

5. The device of claim 4, wherein the first section includes a hook to restrict disengagement of the follower from the pin.

6. The device of claim 1, including an upper and lower base plate for fitment to upper and lower jaws, the base plates being arranged to carry a respective one of the pin or follower.

7. The device of claim 6, wherein the base plates are provided with magnets arranged whereby to exert a magnetic repulsive force between the upper and lower base plates so as to bias the first jaw toward the advanced position relative to the second jaw.

8. The device of claim 7, wherein the base plates are provided with magnets to exert a magnetic attractive force between the plate when the first jaw is advanced relative to the second jaw to assist in maintaining the jaws in that position.

9. The device of claim 6, wherein the follower is in the form of a side plate attached to one of the upper or lower plates.

10. The device of claim 6, further comprising a second follower, wherein the two followers are attached to opposite sides of one of the upper or lower base plates.

11. The device of claim 6, further comprising a second follower and a second pin, wherein the two pins are provided on opposite sides of the other one of the upper or lower base plates for engagement with the associated followers.

12. A mandibular advancement device which comprises both an upper base plate and a lower base plate for fitment with a respective upper and lower jaw, the base plates having co-operating portions that engage during closing of the jaws to cause protrusion of the lower jaw;
wherein the co-operating portions are arranged to enable lateral movement between the base plates during the closing of the lower jaw;
wherein the co-operating portions are located on either side of each of the base plates; and
wherein the device is configured such that the operation of the device is driven by the movement of the jaws as the jaws are closed.

13. The device as claimed in claim 12, wherein one of the co-operating portions is provided with outwardly flared lateral walls to guide relative lateral movement between the jaws.

14. The device as claimed in claim 12, wherein the lateral walls form a tapered channel to guide a first cooperating member, into alignment with a second cooperating member.

15. The device as claimed in claim 12, wherein the co-operating portions contact during closing of the jaws so as to progressively force the lower jaw forward relative to the upper jaw.

16. The device as claimed in claim 12, wherein the co-operating portions are shaped wedges.

17. The device as claimed in claim 12, wherein the upper and lower base plates are provided with opposing magnets to repel the upper and lower base plates from close abutment.

18. The device as claimed in claim 17, wherein the opposed magnets bias the upper and lower base plates in both forward and backward directions to provide controlled repulsion of one base plate relative to the other base plate.

19. The device as claimed in claim 17, wherein the co-operating portions include a specifically shaped portion on one of the base plates, and a guide pin arranged on the other base plate for riding along the specifically shaped portion during closing of the jaws so as to progressively force the lower jaw forward relative to the upper jaw.

20. A mandibular advancement device which comprises both an upper and a lower base plate, wherein the upper base plate incorporates two side followers, which are located on either side of the upper base plate, each side follower including a curved engagement surface that co-operates with the lower plate to enable controlled forward movement of a lower jaw as the upper and lower base plates are moved together;
wherein each of the side followers is shaped so as to push on to a respective opposing adjustment mechanism, located in the opposing outer side of the lower base plate; and
wherein the device is configured such that the operation of the device is driven by the movement of the jaws as the jaws are closed.
